(19) United States
(12) Patent Application Publication
(10) Pub. No.: US 2010/0318193 A1
(43) Pub. Date: Dec. 16, 2010

(54) TOPOGRAPHICALLY ENGINEERED STRUCTURES AND METHODS FOR USING THE SAME IN REGENERATIVE MEDICINE APPLICATIONS

(76) Inventors: Tejal A. Desai, San Francisco, CA (US); Sarah Tao, Cambridge, MA (US); Michael Young, Ipswich, MA (US); Henry J. Klassen, Orange, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/530,013

(22) PCT Filed: Mar. 10, 2008
(86) PCT No.: PCT/US08/56436

§ 371 Date: Jul. 19, 2010

Related U.S. Application Data
(60) Provisional application No. 60/893,775, filed on Mar. 8, 2007.
(60) Provisional application No. 60/911,439, filed on Apr. 12, 2007.

(51) Int. Cl. A61F 2/02

(52) U.S. Cl. 623/23.76; 977/778

(57) ABSTRACT

The present invention provides compositions including a cell contacting surface or film comprising nanotopography of nanofibers, nanotubes, nanochannels, microchannels or microwells, which are capable of enhancing or promoting cell differentiation or cell viability. The compositions are useful as medical implants, including orthopedic implants, dental implants, cardiovascular implants, neurological implants, neurovascular implants, gastrointestinal implants, muscular implants, and ocular implants. The present invention also provides methods of treating a patient in need of such an implant.

### CROSS-REFERENCE

This application claims the benefit of U.S. Provisional Application No. 60/893,775, filed Mar. 8, 2007, and U.S. Provisional Application No. 60/911,439, filed Apr. 12, 2007, which applications are incorporated herein by reference in their entirety.

### BACKGROUND OF THE INVENTION

One of the important challenges to designing better implant materials is to induce tissue growth on or at the implant surface. In a physiological environment, cells respond to nanometric topographies such as fibrous and porous materials formed by components of the extracellular matrix (e.g., callogen, hylauronic acid, laminin, fibronecton, etc.). For example, a number of obstacles remain to the development of a practical ocular implant therapy for the retina remain, including immediate reflux at the time of injection and massive death of donor cells following the standard bolus injection method. While photoreceptor loss is untreatable at present, one of the most promising therapies for late-stage retinal degenerations involves the delivery of stem or progenitor cells to the outer retina.

Several studies have demonstrated improvements in stem and progenitor survival when the cells are delivered to the subretinal space on polymer scaffolds (Tomita et al., Cells. 2005, 23, 1579-88; Warfvinge et al., Arch Opthalmol. 2005, 123, 1385-93; Klassen et al., Prog Retin Eye Res. 2004, 23, 149-181). Compared to cell injection methods, retinal progenitor cells (RPCs) cultured on polymer scaffolds prior to subretinal transplantation in rho−/− mice showed a 10- and 14-fold increase in survival and cell delivery, respectively. However, due to the physical constraint of the sub-retinal space, the use of thick scaffolds (>100 μm) increased the incidence of trauma during the transplantation procedure implicating the need for an alternate approach.

Likewise, in the context of orthopedic and dental implants, the level of bone growth depends on the surface characteristics of the implant. The first event that occurs after the implantation of a biomaterial is the adsorption of proteins from blood and other tissue fluids. Primarily, a hematoma, swelling filled with blood due to a break in the blood vessel, is present between the implant and bone. Cytokines and growth factors stimulate the recruitment of mesenchymal cells which differentiate into osteoblast that are responsible for bone formation. Over time, woven bone matures into lamellar bone which further strengthens the bone-implant interface. Thus, the surface properties play a critical role in long term stability and functionality of the implant.

For example, a large number of implant materials and designs have been used in an attempt to enhance the stability of endosseous implants. In addition to cement-based prosthetics, much attention in recent years has turned to microinterlocked implants, which have microporous surfaces to allow for the ingrowth of tissue. Early work using oxide ceramics showed that a minimum interconnected pore diameter of approximately 100 μm was needed for adequate tissue ingrowth (Hulbert et al., J Biomed Mater Res 1972; 6(5):347-74). It was thought that smaller pore sizes allowed incomplete mineralization of the infiltrating tissue. Subsequent use of metallic implants showed bone ingrowth with pore sizes between 50 and 400 μm (Bobyn et al., Clin Orthop Relat. Res 1980(150):263-70).

However, recent studies have revealed the possibility that much smaller pores may allow bone ingrowth when presented at high density within metal-oxide substrates. For example, nanoporous Ca-P coatings on implants have shown apposition of human bone growth within 2-3 weeks post surgery (Lee et al., J Biomed Mater Res 2001; 55(3):360-7). Osteoblasts cultured on ceramics of different nm-scale textures also exhibit altered morphologies and growth rates (Bogan et al., Biomaterials 1996, 17(2):137-46; Popat et al., J Orthop Res 2006, 24(4):619-27; Popat et al., Biomaterials 2005, 26(22):4516-22; Swan et al., Biomaterials 2005, 26(14):1969-76; Swan et al., J Biomed Mater Res A 2005, 72(3):288-95; Webster et al., Biomaterials 2004, 25(19):4731-9; Webster et al., J Biomed Mater Res A 2003, 67(3):975-80; Webster et al., Biomaterials 2000, 21(17):1803-10). Nonetheless, there are several problems related to dissolution of nanoscale coatings over time, and cracking and separation from the metallic substrate (Bauer et al., Clin Orthop Relat Res 1994, (298):11-8; and Bloebaum et al., Clin Orthop Relat Res 1994, (298):19-26). These studies point to the importance of developing more robust and flexible nanoscale architectures to enhance the apposition of bone from existing bone surfaces and stimulate new bone formation.

Moreover, as shown in recent studies, nanostructures fabricated in metals, semiconductors, and various non-degradable polymers are not ideal for use in biomedical applications, such as orthopedic, dental, or ocular implants. If implanted, many of these materials would permanently remain in the body unless surgically removed. In terms of regenerative medicine, this would mean integration of a fully functional tissue would never be achieved, whereas with microdevices this would result in additional surgery for an inherently difficult retrieval.

This invention described below addresses these needs, as well as others.

### SUMMARY OF THE INVENTION

The present invention provides compositions including a cell contacting surface or film comprising nanotopography of nanofibers, nanotubes, nanochannels, microchannels or microwells, which are capable of enhancing or promoting cell differentiation or cell viability. The compositions are useful as medical implants, including orthopedic implants, dental implants, cardiovascular implants, neurological implants, neurovascular implants, gastrointestinal implants, muscular implants, and ocular implants. The present invention also provides methods of treating a patient in need of such an implant.

The present invention provides a medical implant, including a cell contacting surface or film comprising nanotopography of nanofibers, nanotubes, nanochannels, microchannels or microwells, wherein said nanochannels and microchannels comprise a first and second opening at lateral edges of said cell contacting surface or film, and wherein said nanotopography is capable of enhancing or promoting cell differentiation or cell viability at said cell contacting surface or film.

In some embodiments, the medical implant is an orthopedic implant, a dental implant, a cardiovascular implant, a neurological implant, a neurovascular implant, a gastrointestinal implant, a muscular implant, or an ocular implant. In some embodiments, the cell contacting surface or film expands or unfurls in the presence of a hydrating liquid. In some embodiments, the nanotopography is comprised of poly(DL-lactide-co-glycolide) (PLGA), poly(DL-lactide-co-ε-caprolactone) (DLPLCL), poly(ε-caprolactone) (PCL), collogen, gelatin, agarose, poly(methyl methacrylate), galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above, or combinations thereof. In certain embodiments, the nanotopography is comprised of poly(methyl methacrylate). In some embodiments, the nanotopography is comprised of silicon, titania, zirconia, cobalt-chromium, alumina, silica, barium aluminate, barium titanate, iron oxide, and zinc oxide, or combinations thereof.

In some embodiments, the nanotopography further includes an agent to facilitate cell adhesion and cell growth selected from the group consisting of laminin, fibrin, fibronectin, proteoglycans, glycoproteins, glycosaminoglycans, chemotactic agents, and growth factors. In some embodiments, the nanotopography further includes a bioactive agent for elution to surrounding tissue upon placement of said implant in subject. In some embodiments, the bioactive agent is selected from a growth factor, a steroid agent, an antibody therapy, an antimicrobial agent, an antibiotic, an antiretroviral drug, an anti-inflammatory compound, an antitumor agent and a chemotherapeutic agent. In some embodiments, the nanotopography further comprises cells, such as a stem cell, a retinal progenitor cell, or a neuronal cell. In some embodiments, the nanotopography is capable of limiting cell adhesion and cell growth.

In some embodiments, the nanofibers or nanotubes range in length from about 1 μm to about 70 μm. In some embodiments, the nanofibers or nanotubes range in diameter from about 3 nm to about 300 nm. In some embodiments, the nanotopography comprises nanofibers at a density greater than 100,000,000 nanofibers per square centimeter. In some embodiments, the nanotopography comprises nanotubes at a density greater than 25,000,000 nanotubes per square centimeter. In some embodiments, the nanotubes have a pore diameter range from about 3 nm to about 250 nm.

In some embodiments, the nanotopography ranges in thickness from about 1 μm to about 100 μm. In some embodiments, the nanotopography ranges in thickness from about 2 μm to about 20 μm. In some embodiments, the microwells range in diameter from about 5 μm to about 12 μm. In some embodiments, the nanotopography comprises microwells at a density greater than 150,000 microwells per square centimeter. In some embodiments, the nanochannels range in diameter from about 1 nm to about 1000 nm. In some embodiments, the nanotopography comprises nanochannels at a density greater than 25,000,000 nanochannels per square centimeter. In some embodiments, the microchannels range in diameter from about 1 μm to about 500 μm. In some embodiments, the nanotopography comprises microchannels at a density greater than 150,000 microchannels per square centimeter.

The present invention also provides a method of treating a patient in need of a medical implant, by placing a medical implant into the patient, wherein the medical implant comprises a cell contacting surface or firm comprising nanotopography of nanofibers, nanotubes, nanochannels, microchannels or microwells, wherein said nanochannels and microchannels comprise a first and second opening at lateral edges of said cell contacting surface or film, and wherein said nanotopography is capable of enhancing or promoting cell differentiation or cell viability at said cell contacting surface or film. In some embodiments, the medical implant is an orthopedic implant, a dental implant, a cardiovascular implant, a neurological implant, a neurovascular implant, a gastrointestinal implant, a muscular implant, or an ocular implant. In some embodiments, the cell contacting surface or film expands or unfurls after placement in said patient.

In some embodiments, the nanotopography is comprised of poly(DL-lactide-co-glycolide) (PLGA), poly(DL-lactide-co-ε-caprolactone) (DLPLCL), poly(ε-caprolactone) (PCL), collogen, gelatin, agarose, poly(methyl methacrylate), galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above, or combinations thereof. In certain embodiments, the nanotopography is comprised of poly(methyl methacrylate). In some embodiments, the nanotopography is comprised of silicon, titania, zirconia, cobalt-chromium, alumina, silica, barium aluminate, barium titanate, iron oxide, and zinc oxide, or combinations thereof.

In some embodiments, the nanotopography further comprises an agent to facilitate cell adhesion and cell growth selected from the group consisting of laminin, fibrin, fibronectin, proteoglycans, glycoproteins, glycosaminoglycans, chemotactic agents, and growth factors. In some embodiments, the nanotopography limits cell adhesion and cell growth. In some embodiments, the nanotopography further includes a bioactive agent for elution to surrounding tissue upon placement of said implant in subject. In some embodiments, the bioactive agent is selected from a growth factor, a steroid agent, an antibody therapy, an antimicrobial agent, an antibiotic, an antiretroviral drug, an anti-inflammatory compound, an antitumor agent and a chemotherapeutic agent. In some embodiments, the nanotopography further includes cells, such as a stem cell, a retinal progenitor cell, or a neuronal cell.

In some embodiments, the nanofibers or nanotubes range in length from about 1 μm to about 70 μm. In some embodiments, the nanofibers or nanotubes range in diameter from about 3 nm to about 300 nm. In some embodiments, the nanotopography comprises nanofibers at a density greater than 100,000,000 nanofibers per square centimeter. In some embodiments, the nanotopography comprises nanotubes at a density greater than 25,000,000 nanotubes per square centimeter. In some embodiments, the nanotubes have a pore diameter range from about 3 nm to about 250 nm. In some embodiments, the nanotopography ranges in thickness from about 2 μm to about 20 μm. In some embodiments, the nanotopography ranges in thickness from about 1 μm to about 100 μm. In some embodiments, the microwells range in diameter from about 5 μm to about 12 μm. In some embodiments, the nanotopography comprises microwells at a density greater than 150,000 microwells per square centimeter. In some embodiments, the nanochannels range in diameter from about 1 nm to about 1000 nm. In some embodiments, the nanotopography comprises nanochannels at a density greater than 25,000,000 nanochannels per square centimeter. In some embodiments, the microchannels range in diameter from about 1 μm to about 500 μm. In some embodiments, the nanotopography comprises microchannels at a density greater than 150,000 microchannels per square centimeter.

The present invention also provides a method for transplanting retinal progenitor cells to a subject's retina, by placing a medical implant comprising retinal progenitor cells into the subject's retina, wherein the medical implant comprises a cell contacting surface or firm comprising nanotopography of nanofibers, nanotubes, nanochannels, microchannels or microwells, wherein said nanochannels and microchannels comprise a first and second opening at lateral edges of said cell contacting surface or film, and wherein said nanotopography is capable of enhancing or promoting cell differentiation or cell viability at said cell contacting surface or film, and wherein said placing provides for transplantation of retinal progenitor cells to the subject's retina. In some embodiments, the cell contacting surface or film expands or unfurls after placement in said subject's retina.

In some embodiments, the nanotopography is comprised of a polymer selected from poly(methyl methacrylate), poly(lactine-co-glycolide), ε-caprolactone, and galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above. In some embodiments, the nanotopography is comprised of silicon, titania, zirconia, cobalt-chromium, alumina, silica, barium aluminate, barium titanate, iron oxide, and zinc oxide, or combinations thereof.

In some embodiments, the nanotopography comprises an agent to facilitate cell adhesion and cell growth selected from the group consisting of laminin, fibrin, fibronectin, proteoglycans, glycoproteins, glycosaminoglycans, chemotactic agents, and growth factors. In some embodiments, the nanotopography further comprise a bioactive agent for elution to surrounding tissue upon placement of said implant in subject. In some embodiments, the bioactive agent is selected from a growth factor, a steroid agent, an antibody therapy, an antimicrobial agent, an antibiotic, an antiretroviral drug, an anti-inflammatory compound, an antitumor agent and a chemotherapeutic agent.

In some embodiments, the nanotopography ranges in thickness from about 2 μm to about 20 μm. In some embodiments, the nanofibers or nanotubes range in length from about 1 μm to about 70 μm. In some embodiments, the nanofibers or nanotubes range in diameter from about 3 nm to about 300 nm. In some embodiments, the nanotopography comprises nanofibers at a density greater than 100,000,000 nanofibers per square centimeter. In some embodiments, the nanotopography comprises nanotubes at a density greater than 25,000,000 nanotubes per square centimeter. In some embodiments, the nanotubes have a pore diameter range from about 3 nm to about 250 nm. In some embodiments, the nanotopography ranges in thickness from about 2 μm to about 20 μm. In some embodiments, the nanotopography ranges in thickness from about 1 μm to about 100 μm. In some embodiments, the microwells range in diameter from about 5 μm to about 12 μm. In some embodiments, the nanotopography comprises microwells at a density greater than 150,000 microwells per square centimeter. In some embodiments, the nanochannels range in diameter from about 1 nm to about 1000 nm. In some embodiments, the nanotopography comprises nanochannels at a density greater than 25,000,000 nanochannels per square centimeter. In some embodiments, the microchannels range in diameter from about 1 μm to about 500 μm. In some embodiments, the nanotopography comprises microchannels at a density greater than 150,000 microchannels per square centimeter.

The present invention also provides a medical implant including a cell contacting surface or film comprising nanotopography of nanofibers, nanotubes, nanochannels, microchannels or microwells, wherein said nanochannels and microchannels comprise a first and second opening at lateral edges of said cell contacting surface or film, and wherein said nanotopography is less than about 100 μm in thickness and is capable of enhancing or promoting cell differentiation or cell viability at said cell contacting surface or film. In some embodiments, the medical implant is an orthopedic implant, a dental implant, a cardiovascular implant, a neurological implant, a neurovascular implant, a gastrointestinal implant, a muscular implant, or an ocular implant. In some embodiments, the cell contacting surface or film expands or unfurls in the presence of a hydrating liquid.

In some embodiments, the nanotopography is comprised of poly(DL-lactide-co-glycolide) (PLGA), poly(DL-lactide-co-ε-caprolactone) (DLPLCL), poly(ε-caprolactone) (PCL), collogen, gelatin, agarose, poly(methyl methacrylate), galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above, or combinations thereof. In some embodiments, the nanotopography is comprised of silicon, titania, zirconia, cobalt-chromium, alumina, silica, barium aluminate, barium titanate, iron oxide, and zinc oxide, or combinations thereof.

In some embodiments, the nanotopography further comprises an agent to facilitate cell adhesion and cell growth selected from the group consisting of laminin, fibrin, fibronectin, proteoglycans, glycoproteins, glycosaminoglycans, chemotactic agents, and growth factors. In some embodiments, the nanotopography further comprise a bioactive agent for elution to surrounding tissue upon placement of said implant in subject. In some embodiments, the bioactive agent is selected from a growth factor, a steroid agent, an antibody therapy, an antimicrobial agent, an antibiotic, an antiretroviral drug, an anti-inflammatory compound, an antitumor agent and a chemotherapeutic agent. In some embodiments, the nanotopography is capable of limiting cell adhesion and cell growth. In some embodiments, the nanotopography further includes cells, such as a stem cell, a retinal progenitor cell, or a neuronal cell.

In some embodiments, the nanofibers or nanotubes range in length from about 1 μm to about 70 μm. In some embodiments, the nanofibers or nanotubes range in diameter from about 3 nm to about 300 nm. In some embodiments, the nanotopography comprises nanofibers at a density greater than 100,000,000 nanofibers per square centimeter. In some embodiments, the nanotopography comprises nanotubes at a density greater than 25,000,000 nanotubes per square centimeter. In some embodiments, the nanotubes have a pore diameter range from about 3 nm to about 250 nm. In some embodiments, the nanotopography ranges in thickness from about 2 μm to about 20 μm. In some embodiments, the microwells range in diameter from about 5 μm to about 12 μm. In some embodiments, the nanotopography comprises microwells at a density greater than 150,000 microwells per square centimeter. In some embodiments, the nanochannels range in diameter from about 1 nm to about 1000 nm. In some embodiments, the nanotopography comprises nanochannels at a density greater than 25,000,000 nanochannels per square centimeter. In some embodiments, the microchannels range in diameter from about 1 μm to about 500 μm. In some embodiments, the nanotopography comprises microchannels at a density greater than 150,000 microchannels per square centimeter.

The present invention also provides a medical implant including a a cell contacting surface or film comprising nanotopography of nanofibers, nanotubes, nanochannels, microchannels or microwells, wherein said nanochannels and microchannels comprise a first and second opening at lateral edges of said cell contacting surface or film, and wherein said cell contacting surface or film expands or unfurls in the presence of a hydrating liquid and wherein said nanotopography is capable of enhancing or promoting cell differentiation or cell viability at said cell contacting surface or film. In some embodiments, the medical implant is an orthopedic implant, a dental implant, a cardiovascular implant, a neurological implant, a neurovascular implant, a gastrointestinal implant, a muscular implant, or an ocular implant.

In some embodiments, the nanotopography is comprised of poly(DL-lactide-co-glycolide) (PLGA), poly(DL-lactide-co-ε-caprolactone) (DLPLCL), poly(ε-caprolactone) (PCL), collogen, gelatin, agarose, poly(methyl methacrylate), galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above, or combinations thereof. In some embodiments, the nanotopography is comprised of silicon, titania, zirconia, cobalt-chromium, alumina, silica, barium aluminate, barium titanate, iron oxide, and zinc oxide, or combinations thereof.

In some embodiments, the nanotopography further comprises an agent to facilitate cell adhesion and cell growth selected from the group consisting of laminin, fibrin, fibronectin, proteoglycans, glycoproteins, glycosaminoglycans, chemotactic agents, and growth factors. In some embodiments, the nanotopography further comprise a bioactive agent for elution to surrounding tissue upon placement of said implant in subject. In some embodiments, the bioactive agent is selected from a growth factor, a steroid agent, an antibody therapy, an antimicrobial agent, an antibiotic, an antiretroviral drug, an anti-inflammatory compound, an antitumor agent and a chemotherapeutic agent. In some embodiments, the nanotopography is capable of limiting cell adhesion and cell growth. In some embodiments, the nanotopography further comprises cells, such as a stem cell, a retinal progenitor cell, or a neuronal cell.

In some embodiments, the nanofibers or nanotubes range in length from about 1 μm to about 70 μm. In some embodiments, the nanofibers or nanotubes range in diameter from about 3 nm to about 300 nm. In some embodiments, the nanotopography comprises nanofibers at a density greater than 100,000,000 nanofibers per square centimeter. In some embodiments, the nanotopography comprises nanotubes at a density greater than 25,000,000 nanotubes per square centimeter. In some embodiments, the nanonubes have a pore diameter range from about 3 nm to about 250 nm. In some embodiments, the nanotopography ranges in thickness from about 1 μm to about 100 μm. In some embodiments, the nanotopography ranges in thickness from about 2 μm to about 20 μm. In some embodiments, the microwells range in diameter from about 5 μm to about 12 μm. In some embodiments, the nanotopography comprises microwells at a density greater than 150,000 microwells per square centimeter. In some embodiments, the nanochannels range in diameter from about 1 nm to about 1000 nm. In some embodiments, the nanotopography comprises nanochannels at a density greater than 25,000,000 nanochannels per square centimeter. In some embodiments, the microchannels range in diameter from about 1 μm to about 500 μm. In some embodiments, the nanotopography comprises microchannels at a density greater than 150,000 microchannels per square centimeter.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1, panel A is a schematic fabrication of ultra-thin film PMMA scaffold. PMMA and positive photoresist are first spun on a wafer. The photoresist is exposed to UV light through a mask and developed. Areas of PMMA unmasked by photoresist are then dry etched. The thin-film PMMA is then lifted off the wafer in a single sheet. Panel B is a schematic PMMA scaffold, 6 μm thick, containing pores approximately 11 μm in diameter and spaced 63 μm apart (Scale bar=100 μm).

FIG. 2 shows in vitro adherence of RPCs to PMMA scaffolds. Panel A shows porous PMMA maintains uniform proliferation of RPCs across its surface in culture. Plating 4×105 RPCs on each 10×10 mm scaffold results in proliferating neurospheres from which individual RPCs migrate radially, reaching confluence by day seven in culture. A similar pattern of RPC growth was seen on non-porous scaffolds. Panel B shows the same image frame as panel A under fluorescent illumination revealing that proliferating RPCs are GFP+ (Scale bar=100 μm).

FIG. 3 shows in vivo RPC adherence to porous PMMA and migration into host retina. Micromachined porous PMMA with GFP+RPCs attached to its surface was inserted into the subretinal space of a C57BL/6 host. Panel A is an image of GFP+ cells on the surface of a porous PMMA membrane. The dashed line traces the approximate contour of the membrane. ONL=outer nuclear layer. Panel B is a high magnification image of GFP+ cells migrating into the photoreceptor (ONL) and inner nuclear layer (INL) of the host retina. Blue indicates DAPI stained nuclei of host retinal layers. Scale bar=50 μm.

FIG. 4 shows images of porous PMMA scaffold RPC retention, which leads to enhanced integration and differentiation in host retina. Panel A is an image taken after four weeks in vivo of a non-porous PMMA RPC graft to assess host retina RPC integration. Few, (˜3) per 12 μm section, GFP+ RPCs appear integrated into the INL and ONL region from the non-porous graft. Panels B and C shows that a significantly higher number (˜45) GFP+ RPCs integrate into all host retinal layers from porous grafts. RPCs integrated from porous grafts exhibit a range of retinal neural morphologic differentiation. Panel D is an immunohistochemical analysis showing that RPCs integrated from non-porous grafts failed to express GFAP. Panels E and F are images showing RPCs integrated from porous grafts with morphologies that either spanned all retinal layers or branched radially in the inner plexiform layer expressed GFAP (yellow). Scale bar=50 μm.

FIG. 5 is a graph showing average RPC adherence and survival between non-porous and porous PMMA scaffolds in vivo. The number of RPCs attached to non-porous or porous membranes or integrated into host retina was compared at four weeks in vivo. In non-porous graft conditions (n=5) the average number of RPCs surviving was 1. In 3 of the 5 non-porous transplants no RPC survival was observed. In contrast porous graft transplants (n=5) yielded and average RPC survival of 37.6 per section. In transplants exhibiting RPC survival at four weeks post-implantation, porous scaffolds yielded a 150% increase over non-porous. *p<0.05, Student's t-test.

FIG. 6 are images showing GFP+ RPCs migrating into the retina from the porous micromachined PMMA scaffolds (white lines). Panel A is an image showing GFP+ RPC integrating into the outer nuclear layer (ONL). Panel B is an image showing the same RPC from panel A, co-expressing the photoreceptor marker, recoverin (yellow). Panel C is an image showing RPCs integrating into the ONL express the early neuronal marker, NF-200 (yellow). Panel D is an image showing GFP+ RPCs expressing NF-200 extend a process through a micromachined pore on the PMMA scaffold. Scale bar=50 μm.

FIG. 7 is a series of scanning electron microscope (SEM) images of Nanostructures made from biodegradable polymers. Panel A shows nanostructures made from 50/50 poly(DL-lactide-co-glycolide) (PLGA). Panel B shows nanostructures made from 25/75/75 poly(DL-lactide-co-ε-caprolactone) (25/75 DLPLCL). Panel C shows nanostructures made from 80/20 poly(DL-lactide-co-ε-caprolactone) (80/20 DLPLCL). Panel D shows nanostructures made from poly(ε-caprolactone) (PCL).

FIG. 8 shows nanotube morphology as a function of temperature and time. Panel A shows growth length of nanotube at 130° C. at various time points. Panel B shows growth length of nanotube at 65° C. at various time points. Panel C is an SEM image of free-standing array of nanotube 2.5 μm in length. Panel D is an SEM image of an array of flexible nanofibers 27 μm in length. Panel E is a 20 μm intermittent contact AFM 3D image of nanotube 2.5 μm in length. Panel F is a 1 μm intermittent contact AFM image of a nanofiber array.

FIG. 9 shows PCL nanotube release and degradation. Panel A shows cumulative release of fluorescein and bovine serum albumin from PCL nanotubes. Panel B shows an SEM image of PCL nanotubes after a degradation period of 7 weeks.

FIG. 10 Potential applications of PCL nanotubes. Panel A is an SEM image of PCL nanofiber patterns (square outline) atop an array of nanotubes. Insert: magnification of the nanofiber/nanotube interface is depicted by arrows. Panel B is an SEM image of fibroblast cells interacting PCL nanotube surface after 3 days in culture. Arrows point to examples of individual cells.

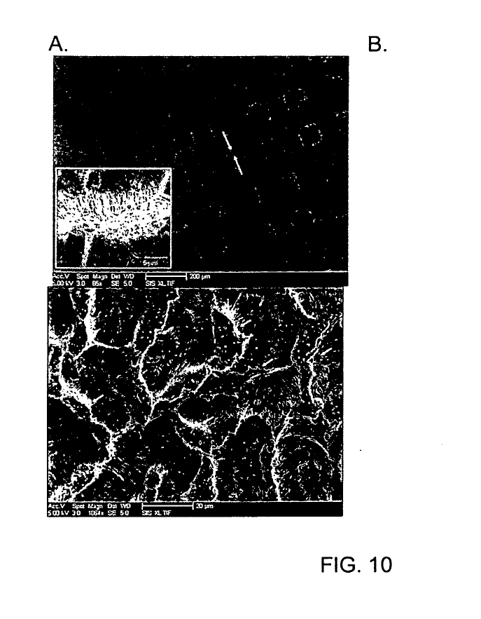

### DETAILED DESCRIPTION OF THE INVENTION

The present invention provides compositions including a cell contacting surface or film comprising nanotopography of nanofibers, nanotubes, nanochannels, microchannels or microwells, which are capable of enhancing or promoting cell differentiation or cell viability. The compositions are useful as medical implants, including orthopedic implants, dental implants, cardiovascular implants, neurological implants, neurovascular implants, gastrointestinal implants, muscular implants, and ocular implants. The present invention also provides methods of treating a patient in need of such an implant.

Before the present Invention described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the compound” includes reference to one or more compounds and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### I. DEFINITIONS AND ABBREVIATIONS

The abbreviations used herein generally have their conventional meaning within the chemical and biological arts.

The term “autologous cells”, as used herein, refers to cells which are person's own genetically identical cells.

The term “heterologous cells”, as used herein, refers to cells which are not person's own and are genetically different cells.

The term “stem cells”, as used herein, refers to cells capable of differentiation into other cell types, including those having a particular, specialized function (i.e., terminally differentiated cells). Stem cells can be defined according to their source (adult/somatic stem cells, embryonic stem cells), or according to their potency (totipotent, pluripotent, multipotent and unipotent).

The term “unipotent”, as used herein, refers to cells can produce only one cell type, but have the property of self-renewal which distinguishes them from non-stem cells.

The term, “multipotent”, or “progenitor”, as used herein, refers to cells which can give rise to any one of several different terminally differentiated cell types. These different cell types are usually closely related (e.g. blood cells such as red blood cells, white blood cells and platelets). For example, retinal progenitor cells (RPCs) include cells that differentiate into any one of the five types of mature retinal cells (e.g., photoreceptors, bipolar cells, horizontal cells, amacrine cells, and ganglion cells).

The term “pluripotent”, as used herein, refers to cells that give rise to some or many, but not all, of the cell types of an organism. Pluripotent stem cells are able to differentiate into any cell type in the body of a mature organism, although without reprogramming they are unable to de-differentiate into the cells from which they were derived. As will be appreciated, “multipotent”/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells.

The term “totipotent”, as used herein, refers to fertilized oocytes, as well as cells produced by the first few divisions of the fertilized egg cell (e.g., embryos at the two and four cell stages of development). Totipotent cells have the ability to differentiate into any type of cell of the particular species. For example, a single totipotent stem cell could give rise to a complete animal, as well as to any of the myriad of cell types found in the particular species (e.g., humans).

The term “anti-aging environment”, as used herein, is an environment which will cause a cell to dedifferentiate, or to maintain its current state of differentiation. For example, in an anti-aging environment, a retinal progenitor cells would either maintain its current state of differentiation, or it would dedifferentiate into a satellite cell.

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “growth environment” is an environment in which stem cells will proliferate in vitro. Features of the environment include the medium in which the cells are cultured, and a supporting structure (such as a substrate on a solid surface) if present.

The term “differentiation factor”, as used herein, refers to a molecule that induces a stem cell to commit to a particular specialized cell type.

The term “regenerative capacity”, as used herein, refers to conversion of stem cell into dividing progenitor cell and differentiated tissue-specific cell.

The term “rejuvenation”, as used herein, refers to changing the regenerative responses of a stem cell such that the stem cell successfully or productively regenerates tissues in organs even if such organs and tissues are old and the stem cells are old.

“Composition of the invention,” as used herein refers to the compositions discussed herein, pharmaceutically acceptable salts and prodrugs of these compositions.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The term “excipients” is conventionally known to mean carriers, diluents and/or vehicles used in formulating drug compositions effective for the desired use.

### II. INTRODUCTION

The present invention is based on the observation that a surface or film having a nanotopography of nanofibers, or nanotubes, nanochannels, microchannels or microwells, that are optionally biodegradable, provide a favorable template for cell growth and differentiation and supported higher cell adhesion, proliferation and viability, as well as localized delivery of cells or therapeutic agents to the implant site, while not causing adverse immune response under in vivo conditions. In addition, the optional use of biodegradable materials translates to a potential for controlled release of trophic factors or therapeutic agents as well as a means to eliminate surgical removal of implants. As such, the inventors have found that the optional biodegradable nanostructure surfaces are capable of delivering drugs locally while providing a favorable biological integration.

The present invention is based on the observation that ultra-thin polymer scaffolds, such as poly(methyl methacrylate) (PMMA) scaffolds, which contain specific topographies provide a means to increase the ease of delivery and reduce the risk of trauma while allowing the scaffold to rest against the retina thereby enhancing potential integration with the host.

For example, for orthopedic implants, the subject implants are capable of simultaneously enhancing osseointegration while also delivering therapeutics which may enhance bone growth or fight off infection. In the case of vascular stents, the nanostructured surface coating is capable of not only delivering anti-inflammatory drugs but also preventing formation of fibrous scar tissue on the stent surface. Moreover, with respect to ocular implants, there are at least two major advantages to using polymer scaffolds, such as PMMA, with adhesive properties instead of bolus injections for transplantation of stem cells, such as RPCs, into the subretinal space, which include increased cell survival and delivery localization to specific retinal regions. Earlier studies attempting to deliver brain-derived neurons into the subretinal space resulted in approximately 90% cell death during the injection process alone. The use of polymer scaffolds for the delivery of stem cells provides a nine-fold increase in cell survival and a sixteen-fold increase in cell delivery. For the treatment of a retinal degenerative disorder like age-related macular degeneration, where loss of retinal neurons occurs primarily in the macula region, placement of RPC seeded PMMA grafts allows for localized cell replacement.

The invention is now described in greater detail.

### III. METHODS AND COMPOSITIONS

As noted above, the present invention provides compositions including optionally biodegradable cell contacting surface or film having a nanotopography of nanotubes, nanofibers, nanochannels, microchannels, or microwells, and medical implants including the optionally biodegradable nanotopography surfaces for use in treating a patient in need of a medical implant. Exemplary medical implants include, but are not limited to, an orthopedic implant, a dental implant, a cardiovascular implant, a neurological implant, a neurovascular implant, a gastrointestinal implant, a muscular implant, an ocular implant, and the like.

In some embodiments, the surface or film expands or unfurls in the presence of a hydrating liquid, such as water present in an insertion site of a subject. By “expands” is meant that surface or film becomes larger in size or volume as a result surrounding liquid hydrating the surface or film. By “unfurl” is meant that the surface or film is unrolled, unfolded, or spread out as a result surrounding liquid hydrating the surface or film

Exemplary surfaces and films can be fabricated from a variety of suitable materials that provide the optional desirable biodegradable quality as well as the ability to form the desired nanotopography of nanotubes, nanofibers, nanochannels, microchannels, and microwells. Exemplary materials include, but are not limited to, biodegradable or bioerodible polymer, such as poly(DL-lactide-co-glycolide) (PLGA), poly(DL-lactide-co-ε-caprolactone) (DLPLCL), or poly(ε-caprolactone) (PCL), as well as natural biodegradable polymers, such as collogen, gelatin, agarose, and the like. PLGA is a bulk-eroding copolymer of polylactide (PLA) and polyglycolide (PGA), where the ingress of water is faster than the rate of degradation. In this case, degradation takes place throughout the whole of the polymer sample, and proceeds until a critical molecular weight is reached, at which point degradation products become small enough to be solubilized. At this point, the structure starts to become significantly more porous and hydrated. The combination of fast-resorbing PGA and slow-resorbing PLA allows PLGA copolymers to have a resoprtion rate of approximately 6 weeks. Fast-resorbing PLGA polymers display high shrinkage, which may not present a stable substrate for cells to lay down extracellular matrix. In addition, the production of acidic degradation species by fast-resorbing polymers can compromise tissue repair.

In addition, the nanotopography can be fabricated from a variety of suitable metal oxides selected from the group consisting of alumina, titania, Ti6Al4V, nickel, zirconia, cobalt-chromium, alumina, silica, barium aluminate, barium titanate, iron oxide, and zinc oxide, as well as shape memory alloys, such as nitinol, or combinations thereof. In certain embodiments, the nanotubes are fabricated of titania.

The nanotopography surface can be fabricated in any number of well known methods. In an exemplary method the biodegradable nanostructure is formed by utilizing a hot melt/wetting technique in which a biodegradable polymer composition is brought to melting temperature or past glass transition temperatures while in contact with a suitable template. In general, the biodegradable polymer composition is heated to a temperature of up to 60° C. to about 140° C. In certain embodiments, the biodegradable polymer composition is heated to about 65° C. In other embodiments, the biodegradable polymer composition is heated to about 130° C. The biodegradable polymer composition is heated to a suitable temperature for a period of time that allows for formation of the nanofiber or nanotube structures of desirable length.

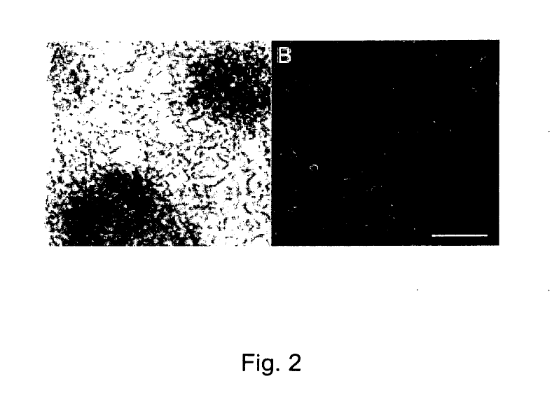

In general, the length of the nanofiber or nanotube structures is a function of the period of time at which the composition is heated as well as the temperature as exemplified in FIG. 2, panels A and B. For example, the biodegradable polymer composition can be heated for a period of time ranging from about 1 minute to about 400 minutes or more, including about 2 minutes to about 390 minutes, about 5 minutes to about 380 minutes, about 10 minutes to about 370 minutes, about 20 minutes to about 360 minutes, about 30 minutes to about 360 minutes, about 40 minutes to about 350 minutes, about 50 minutes to about 340 minutes, 60 minutes to about 330 minutes, about 70 minutes to about 320 minutes, about 80 minutes to about 310 minutes, about 90 minutes to about 300 minutes, about 100 minutes to about 290 minutes, 120 minutes to about 270 minutes, about 140 minutes to about 250 minutes, about 160 minutes to about 230 minutes, about 180 minutes to about 210 minutes, and the like.

In some embodiments, the delivery of the bioactive compounds is by elution from the nanochannels and microchannels. In such embodiments, the nanochannels and microchannels include high molecular weight bioactive compounds and the constraints of the structure, such as diameter of the nanochannels and microchannels, controls the elution rate of the bioactive compound, thereby resulting in a zero order drug delivery kinetic. In some embodiment, the medical devices include combinations of topographical structures, such as, for example, microwells for delivery of cells and nanochannels or microchannels for delivery of bioactive compounds.

In some embodiments, the biodegradable polymer composition is heated to about 65° C. for a period of about 15 minutes to about 80 minutes. In other embodiments, the biodegradable polymer composition is heated to about 130° C. for a period of about 15 minutes to about 200 minutes.

The ability to fabricate arrays of nanotubes and nanofibers from biodegradable polymers using this fast and inexpensive method of template synthesis holds many advantages over the electrospinning and combination templating methods previously described. First, the method is simple and there is no need for specialized equipment or set-up. Second, the general structure of the nanotubes can be controlled by the template design itself. While constricted to a single template design, it is still possible to control nanotube length and to a degree, nanotube diameter. This allows for the fabrication of aligned arrays of free standing nanotubes or flexible nanofibers rather than an unordered surface. Third, although the presence of densely packed nanotubes and nanofibers of biodegradable polymers, such as PCL, does decrease the wettability of the surface, the added roughness does not cause drastic changes to form super hydrophobic or super hydrophilic surfaces. Therefore, similar chemical modification processes may be used to alter both smooth and nanotube/fiber surfaces for further control over protein and cell adhesion in biomedical applications.

Furthermore, the use of template synthesis allows the nanotopography to be loaded with drug molecules without the use of organic solvents, which is especially important in the case of protein and peptide therapeutics. Therefore, the high surface area to volume ratio of nanotube/fiber arrays made of biodegradable polymers, such as PCL, would ensure biodegradation and resorption as well as provide a means for delivering controlled doses of bioactive agents locally at the implant site or the site of regeneration.

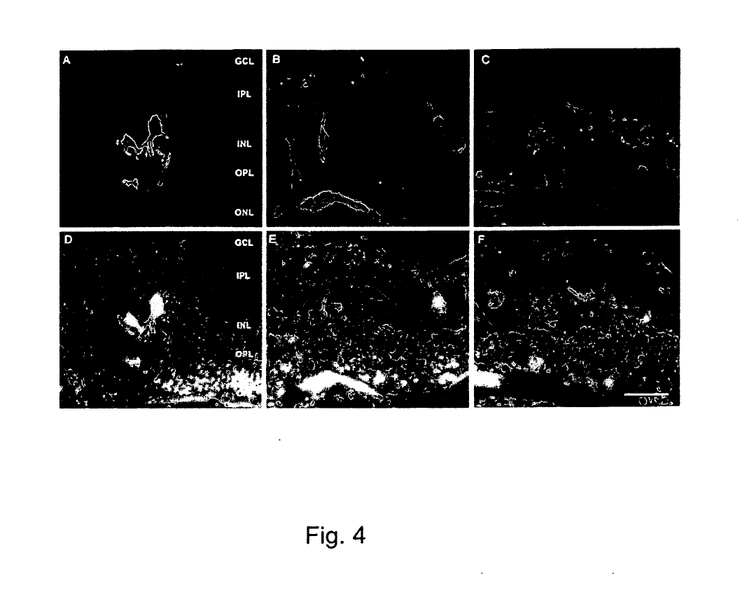

In addition, templating methods for fabricating nanotubes and nanofibers from biodegradable polymers may be combined with patterning at the micron level to create biointerfaces with hierarchical nano- and microarchitecture. For example, FIG. 4, panel A shows an example of nanofibers patterned in the shape of an 80 μm square atop an array of free-standing nanotubes. Other results show that cell morphology may be controlled by subcellular interactions with the nanotube substrates (FIG. 4, panel B). The ability to design hierarchical structures on the nano- and micro-level will allow for even more sophisticated constructs capable of controlling delivery of therapeutics and cellular responses. The capability to control cell responses at both the nano- and microscale using material properties will be useful not only in the regeneration of hard and soft tissues, but also in determining the biointegration of implantables such as microdevices, stents, orthopedic implants, and biosensors.

In general, the nanotubes or nanofibers are fabricated to have a diameter ranging from about 3 nm to about 300 nm, including about 10 nm to about 250 nm, about 20 nm to about 225 nm, about 30 nm to about 200 nm, about 50 nm to about 190 nm, about 60 nm to about 180 nm, about 70 nm to about 170 nm, about 80 nm to about 160 nm, and about 90 nm to about 150 nm. In some embodiments, the nanofibers are fabricated at a density greater than at least about 100,000,000 nanofibers per square centimeter or more, including at least about 200,000,000 nanofibers per square centimeter, and at least about 300,000,000 nanofibers per square centimeter. In some embodiments the nanotubes are fabricated at a density greater than at least about 25,000,000 nanotubes per square centimeter, including at least about 50,000,000 nanotubes per square centimeter, and at least about 75,000,000 nanotubes per square centimeter.

In general, the nanotubes or nanofibers are fabricated to have a length ranging from about 1 μm to about 70 μm, including about 2 μm to about 60 μm, about 3 μm to about 50 μm, about 4 μm to about 40 μm, about 5 μm to about 30 μm, about 6 μm to about 25 μm, about 7 μm to about 24 μm, about 8 μm to about 23 μm, about 10 μm to about 20 μm, about 12 μm to about 18 μm, and about 14 μm to about 16 μm. In an exemplary embodiment, the nanotubes have a length of about 10 μm.

In general, the nanotubes are fabricated to have pores range in diameter from about 3 nm to about 250 nm, including 4 nm to about 225 nm, including 5 nm to about 200 nm, including 6 nm to about 175 nm, including 7 nm to about 150 nm, including 8 nm to about 125 nm, including 9 nm to about 100 nm, including 10 nm to about 75 nm, including 11 nm to about 70 nm, including 12 nm to about 65 nm, including 13 nm to about 60 nm, including 14 nm to about 50 nm, including 15 nm to about 45 nm, about 20 nm to about 40 nm, about 22 nm to about 38 nm, about 24 nm to about 36 nm, about 26 nm to about 34 nm, about 28 nm to about 32 nm, and about 29 nm to about 31 nm. In an exemplary embodiment, the pores have in diameter of about 20 nm to about 40 nm.

In general, the microwells are fabricated to have a first and second opening extending between the lateral edges of cell contacting surface or film and have a diameter ranging from about 1 μm to about 100 μm, including about 2 μm to about 90 μm, about 3 μm to about 80 μm, about 4 μm to about 70 μm, about 5 μm to about 60 μm, about 6 μm to about 50 μm, about 7 μm to about 40 m, about 8 μm to about 30 μm, and about 7 μm to about 20 μm. In some embodiments, the microwells are fabricated to have a diameter ranging from about 1 μm to about 12 μm. In some embodiments, the microwells are fabricated at a density greater than at least about 150,000 microwells per square centimeter or more, including at least about 200,000 microwells per square centimeter, and at least about 300,000 microwells per square centimeter.

In general, the nanochannels are fabricated to have a first and second opening extending between the lateral edges of cell contacting surface or film and have a diameter from about 1 nm to about 2000 nm, including 10 nm to about 1500 nm, about 20 nm to about 1000 nm, about 30 nm to about 500 nm, about 40 nm to about 400 nm, about 50 nm to about 300 nm, about 60 nm to about 200 nm, and about 70 nm to about 100 nm. In some embodiments, the nanochannels are fabricated at a density greater than at least about 25,000,000 nanochannels per square centimeter, including at least about 75,000,000 nanochannels per square centimeter, and at least about 100,000,000 nanochannels per square centimeter.

In general, the microchannels are fabricated to have a diameter from about 1 μm to about 1000 μm, including 10 μm to about 500 μm, about 20 nm to about 1000 nm, about 30 nm to about 500 nm, about 40 nm to about 400 nm, about 50 nm to about 300 nm, about 60 nm to about 200 nm, and about 70 nm to about 100 nm. In some embodiments, the microchannels are fabricated at a density greater than at least about 150,000 microchannels per square centimeter, including at least about 200,000 microchannels per square centimeter, and at least about 300,000 microchannels per square centimeter.

In general, the surface or film is fabricated to have a thickness ranging from about 2 μm to about 500 μm, including about 5 μm to about 400 μm, about 10 μm to about 300 μm, about 20 μm to about 100 μm, about 30 μm to about 70 μm, and about 40 μm to about 60 μm. In certain embodiments, the polymer scaffolds have a thickness ranging from about 2 μm to about 20 μm, including about 3 μm to about 19 μm, about 4 μm to about 18 μm, about 5 μm to about 17 μm, about 6 μm to about 16 μm, about 7 μm to about 15 μm, about 8 μm to about 14 μm, about 9 μm to about 13 μm, and about 10 μm to about 12 μm. In an exemplary embodiment, the polymer scaffolds have in thickness of about 6 μm.

In certain embodiments, the nanotopography of the medical implants further include advantageous biological agents and additives to impart, for example, additional osteoinductive and osteoconductive properties to the surface-modified implants. This may be particularly useful for implants of the present invention that are bone implants. In an exemplary embodiment, one or more biological agents or additives may be added to the implant before implantation. The biological agents and additives may be adsorbed onto and incorporated into the biodegradable nanostructure coated surface, by dipping the implant into a solution or dispersion containing the agents and/or additives, or by other means recognized by those skilled in the art. In some embodiments, the biodegradable nanostructure will release the adsorbed biological agents and additives in a time-controlled fashion. In this way, the therapeutic advantages imparted by the addition of biological agents and additives may be continued for an extended period of time.

The biological agents or additives may be in a purified form, partially purified form, recombinant form, or any other form appropriate for inclusion in the surface-modified medical implant. It is desirable that the agents or additives be free of impurities and contaminants. Exemplary agents to facilitate cell adhesion and cell grow include laminin, fibrin, fibronectin, proteoglycans, glycoproteins, glycosaminoglycans, chemotactic agents, and growth factors, and the like.

For example, growth factors may be included in the nanotopography of the implant to encourage bone or tissue growth. Non-limiting examples of growth factors that may be included are platelet derived growth factor (PDGF), transforming growth factor β (TGF-β), insulin-related growth factor-I (IGF-I), insulin-related growth factor-II (IGF-II), fibroblast growth factor (FGF), beta-2-microglobulin (BDGF II), and bone morphogenetic factors. Bone morphogenetic factors are growth factors whose activity is specific to bone tissue including, but not limited to, proteins of demineralized bone, demineralized bone matrix (DBM), and in particular bone protein (BP) or bone morphogenetic protein (BMP). Osteoinductive factors such as fibronectin (FN), osteonectin (ON), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, transforming growth factor (TGF-beta), insulin-like growth factor (IGF-1), platelet derived growth factors (PDGF), and fibroblast growth factors (FGF, bFGF, etc.) also may be included in the surface-modified implant.

Still other examples of biological agents and additives that may be incorporated in the nanotopography of the medical implant are biocidal/biostatic sugars such as dextran and glucose; peptides; nucleic acid and amino acid sequences such as leptin antagonists, leptin receptor antagonists, and antisense leptin nucleic acids; vitamins; inorganic elements; co-factors for protein synthesis; antibody therapies, such as Herceptin®, Rituxan®, Myllotarg®, and Erbitux®; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; bone digestors; antitumor agents and chemotherapeutics such as cis-platinum, ifosfamide, methotrexate, and doxorubicin hydrochloride; immuno-suppressants; permeation enhancers such as fatty acid esters including laureate, myristate, and stearate monoesters of polyethylene glycol; bisphosphonates such as alendronate, clodronate, etidronate, ibandronate, (3-amino-1-hydroxypropylidene)-1,1-bisphosphonate (APD), dichloromethylene bisphosphonate, aminobisphosphonatezolendronate, and pamidronate; pain killers and anti-inflammatories such as non-steroidal anti-inflammatory drugs (NSAID) like ketorolac tromethamine, lidocaine hydrochloride, bipivacaine hydrochloride, and ibuprofen; antibiotics and antiretroviral drugs such as tetracycline, vancomycin, cephalosporin, erythromycin, bacitracin, neomycin, penicillin, polymycin B, biomycin, chloromycetin, streptomycin, cefazolin, ampicillin, azactam, tobramycin, clindamycin, gentamicin, and aminoglycocides such as tobramycin and gentamicin; and salts such as strontium salt, fluoride salt, magnesium salt, and sodium salt.

The optionally biodegradable surfaces or films having the nanotopography with or without adhesion-promoting peptides and/or other biological agents can be compacted and/or structured and used alone to form an implant. Alternatively, a structured substrate can be coated with a composition comprising the surfaces or films having the nanotopography with or without adhesion-promoting peptides. Substrates include any conventional substrates for medical implants or for other types of implants known in the art.

Also provided is a method of treating a patient in need of a medical implant comprising the steps of selecting the medical implant wherein the implant comprises the cell contacting surface or film having the nanotopography that is capable of enhancing or promoting cell differentiation or cell viability and placing the implant into the patient. Exemplary implants include, orthopedic implants, dental implants, cardiovascular implants, such as a pacemaker, neurological implants, neurovascular implants, gastrointestinal implants, muscular implants, ocular implants, and the like. In this embodiment of the invention the term “selecting” means, for example, purchasing, choosing, or providing the implant rather than preparing the implant.

The method of the present invention can be used for both human clinical medicine and veterinary applications. Thus, the patient can be a human or, in the case of veterinary applications, can be a laboratory, agricultural, domestic, or wild animal. The present invention can be applied to animals including, but not limited to, humans, laboratory animals such as monkeys and chimpanzees, domestic animals such as dogs and cats, agricultural animals such as cows, horses, pigs, sheep, goats, and wild animals in captivity such as bears, pandas, lions, tigers, leopards, elephants, zebras, giraffes, gorillas, dolphins, and whales.

In another embodiment, a method for enhancing osseointegration of an orthopedic implant is provided. The method comprises the steps of selecting the orthopedic implant wherein the implant comprises the cell contacting surface or film having the nanotopography that is capable of enhancing or promoting cell differentiation or cell viability and placing the implant into a patient. In this embodiment of the invention the term “selecting” means, for example, purchasing, choosing, or providing the implant rather than preparing the implant. The patient can be a human or, in the case of veterinary applications, can be a laboratory, agricultural, domestic, or wild animal.

Enhancement of osseointegration is increased osseointegration compared to that obtained with conventional implant materials. Enhanced osseointegration can be demonstrated by increased osteoblast adhesion, increased osteoblast proliferation, increased calcium deposition, enzyme activity assays, or by any other art-recognized technique used to detect osseointegration.

In yet another embodiment a method of preparing a medical implant is provided. The method comprises the step of forming a composition comprising a cell contacting surface or film having the nanotopography that is capable of enhancing or promoting cell differentiation or cell viability. The method can further comprise the step of coating a substrate with the surface or film having the nanotopography. The surface or film can be a composition containing the nanotopography alone, a nanocomposite composition, a nanocomposite composition containing an adhesion-promoting peptide, or any other composition containing nanotopography that is suitable for use in accordance with the present invention.

Use in Ocular Implants

As noted above, the present invention provides methods and compositions for use in transplanting cells, such as stem cells, retinal progenitor cells, or neuronal cells, to the eye of a subject for regenerative medicine in the eye and for treatment of ocular disease. For example, a suitable thin-film that is atraumatic can be fabricated by micro-machining with suitable surface nanotopography to allow for surface area for cell attachment, while providing a means for transport across the scaffold. Exemplary surfaces or films can be designed with an overall thinness in order to increase the ease of delivery and reduce risk of trauma during transplantation to the eye. In certain embodiments, the size of the thin polymer film will be such that it is thinner than the interstitial gap of the subretinal space.

The polymer film may be fabricated of any polymer material that provides the desirable properties described here, such as nanotopography, porosity, and size. Suitable polymers include, but are not limited to, poly(methyl methacrylate), poly(lactine-co-glycolide), ε-caprolactone, and galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above. In an exemplary embodiment, the polymer scaffolds are fabricated using poly(methyl methacrylate).

In some embodiments, the polymer films are fabricated from a biodegradable or bioerodible polymer, such as PLGA or PCL. PLGA is a bulk-eroding copolymer of polylactide (PLA) and polyglycolide (PGA), where the ingress of water is faster than the rate of degradation. In this case, degradation takes place throughout the whole of the polymer sample, and proceeds until a critical molecular weight is reached, at which point degradation products become small enough to be solubilized. At this point, the structure starts to become significantly more porous and hydrated. The combination of fast-resorbing PGA and slow-resorbing PLA allows PLGA copolymers to have a resoprtion rate of approximately 6 weeks. Fast-resorbing PLGA polymers display high shrinkage, which may not present a stable substrate for cells to lay down extracellular matrix. In addition, the production of acidic degradation species by fast-resorbing polymers can compromise tissue repair.

In general, the polymer films are fabricated to have a thickness ranging from about 2 μm to about 500 μm, including about 5 μm to about 400 μm, about 10 μm to about 300 μm, about 20 μm to about 100 μm, about 30 μm to about 70 μm, and about 40 μm to about 60 μm. In certain embodiments, the polymer scaffolds have a thickness ranging from about 2 μm to about 20 μm, including about 3 μm to about 19 μm, about 4 μm to about 18 μm, about 5 μm to about 17 μm, about 6 μm to about 16 μm, about 7 μm to about 15 μm, about 8 μm to about 14 μm, about 9 μm to about 13 μm, and about 10 μm to about 12 μm. In an exemplary embodiment, the polymer scaffolds have in thickness of about 6 μm.

The polymer film is fabricated to include a surface nanotopography, such as nanotubes, nanofibers, microchannels, and microwells that provides for cell adhesion, cell growth, and promote cell differentiation. By including a surface nanotopography and architecture the polymer film will better mimic in vivo structures, such as extra-cellular matrices, thereby influencing host response. For example, in some embodiments, the polymer scaffolds can be fabricated to include pores and microchannel structures that provide laminar organization and structural guidance for axonal growth.

In certain embodiments, the polymer scaffolds further include a plurality of pores as described above. The general, the pores range in diameter from about 5 μm to about 12 μm, including 6 μm to about 15 μm, about 7 μm to about 14 μm, about 8 μm to about 13 μm, about 9 μm to about 12 μm, and about 10 μm to about 11 μm. In an exemplary embodiment, the pores have in diameter of about 11 μm.

In certain embodiments, the nanotopography include an agent to facilitate cell adhesion and cell growth. Suitable agents to facilitate cell adhesion and cell growth include, but are not limited to, laminin, fibrin, fibronectin, proteoglycans, glycoproteins, glycosaminoglycans, chemotactic agents, and growth factors.

In certain embodiments, the nanotopography include a bioactive agent that can be released to the surrounding tissue of the eye. Examples of bioactive agents that may be delivered by the polymer scaffolds of the invention include drugs, medicaments, antibiotics, antibacterials, antiproliferatives, neuroprotectives, anti-inflammatories (steroidal and non-sterodial), growth factors, neurotropic factors, antiangiogenics, thromobolytics or genes. More specifically, the one or more bioactive agents may be selected from thrombin inhibitors; anti thrombogenic agents; thrombolytic agents; fibrinolytic agents; vasospasm inhibitors; calcium channel blockers; vasodilators; antihypertensive agents; antimicrobial agents, antifungals, and antivirals; inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives, including antiangiogenesis agents; anticancer chemotherapeutic agents; anti-inflammatories; non-steroidal anti-inflammatories; antiallergenics; anti-proliferative agents; decongestants; miotics and anti-cholinesterase; antineoplastics; immunological drugs; hormonal agents; immunosuppressive agents, growth hormone antagonists, growth factors; inhibitors of angiogenesis; dopamine agonists; radiotherapeutic agents; peptides; proteins; enzymes; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and combinations thereof.

The surface or film having the nanotopography and including the cells, e.g., stem cells, retinal progenitor cells, or neuronal cells, may be administered intraocularly to a variety of locations depending on the type of disease to be treated, prevented, or, inhibited, and the extent of disease. Examples of suitable locations include the retina (e.g., for retinal diseases), the vitreous, or other locations in or adjacent to the eye.

Briefly, the human retina is organized in a fairly exact mosaic. In the fovea, he mosaic is a hexagonal packing of cones. Outside the fovea, the rods break up the close hexagonal packing of the cones but still allow an organized architecture with cones rather evenly spaced surrounded by rings of rods. Thus in terms of densities of the different photoreceptor populations in the human retina, it is clear that the cone density is highest in the foveal pit and falls rapidly outside the fovea to a fairly even density into the peripheral retina (see Osterberg, G. (1935) Topography of the layer of rods and cones in the human retina. Acta Ophthal. (suppl.) 6, 1-103; see also Curcio, C. A., Sloan, K. R., Packer, O., Hendrickson, A. E. and Kalina, R. E. (1987) Distribution of cones in human and monkey retina: individual variability and radial asymmetry. Science 236, 579-582). Access to desired portions of the retina, or to other parts of the eye may be readily accomplished by one of skill in the art (see, generally Medical and Surgical Retina: Advances, Controversies, and Management, Hilet Lewis, Stephen J. Ryan, Eds., medical illustrator, Timothy C. Hengst. St. Louis: Mosby, c1994. xix, 534; see also Retina, Stephen J. Ryan, editor in chief, 2nd ed., St. Louis, Mo.: Mosby, c1994. 3 v. (xxix. 2559 p.).

As noted above, this invention relates to the discovery that surfaces and films having the nanotopography of nanofibers, nanotubes, nanochannels, microchannels or microwells, and including cells, such as stem cells, retinal progenitor cells, or neuronal cells, may be useful for treating ocular diseases and disorders affecting, for example, the retina, cornea, etc., such neurodegenerative retinal diseases as described herein. Exemplary ocular disorders include, but are not limited to, retinopathy, hypertensive retinopathy, diabetic retinopathy, occlusive retinopathy, retinal degeneration, retinal degeneration caused by injury, retinal degeneration caused by a genetic disorder, retinal degeneration by retinitis pigmentosa, and retinal degeneration caused by age related macular degeneration, or is caused by elevated intraocular pressure or an optic neuropathy, or involves retinal cell damage.

A subject in need of such treatment may be a human or non-human primate or other animal and who has developed symptoms of a retinal disease or who is at risk for developing a neurodegenerative disease. Treating such a subject is understood to encompass preventing further cell death, or replacing, augmenting, repairing, or repopulating damaged tissue and cells by administering retinal stem cells. Preferably, the retinal stem cells are administered to a subject in need thereof prior to the end-stage of a neurodegenerative disease, and preferably at a time point prior to initiation of ocular disease or at a time point that will prevent, slow, or impair further the progression of the ocular disease (that is, for example, soon after an initial diagnosis has been made). By way of example, a diagnosis of macular degeneration can be made at early stages of the disease. According to the present invention, introduction of retinal stem cells at the time of diagnosis may delay, prevent, impair, or inhibit further neurodegeneration of retinal neuronal cells by preventing photoreceptor cell death.

### V. KITS

Kits for use in connection with the subject invention are also provided. The above-described surfaces or films having nanotopography of nanofibers, nanotubes, nanochannels, microchannels or microwells, that are capable of enhancing or promoting cell differentiation or cell viability at said cell contacting surface or film, as well as reagents for carrying out the methods described herein, can be provided in kits, with suitable instructions in order to conduct the methods as described above. The kit will normally contain in separate containers the polymer scaffolds or materials necessary for fabricating the polymer scaffolds. Instructions (e.g., written, tape, VCR, CD-ROM, etc.) for carrying out the methods usually will be included in the kit. The kit can also contain, depending on the particular method, other packaged reagents and materials (i.e. buffers and the like).

The instructions are generally recorded on a suitable recording medium. For example, the instructions may be printed on a substrate, such as paper or plastic, etc. As such, the instructions may be present in the kits as a package insert, in the labeling of the container of the kit or components thereof (e.g., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed from or from where the instructions can be downloaded.

Still further, the kit may be one in which the instructions are obtained are downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

The following methods and materials were used in the Examples below.

PMMA Thin Film Scaffold Fabrication

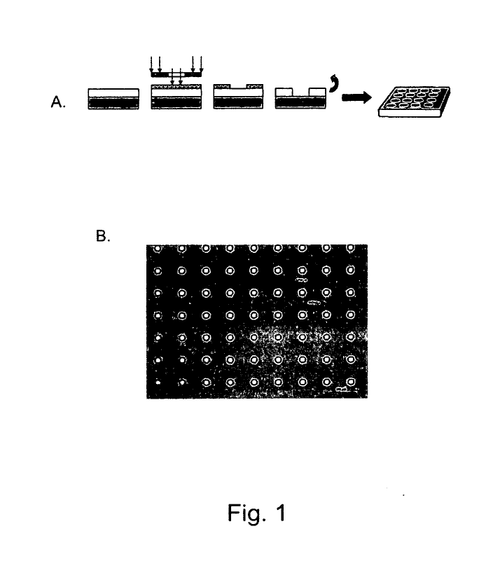

Ultra-thin film PMMA scaffolds were fabricated utilizing a two-step process of photolithography and reactive ion etching (FIG. 1, panel A) (Tao et al., J. Contro. Release. 2003, 88, 215-28). The polymer scaffolds were prepared on Radio Corporation of America (RCA) cleaned silicon <111> p-type wafers (Addison-Engineering, San Jose, Calif.) coated with a water-soluble lift-off layer. The wafers were then double-coated with PMMA (950,000 MW, Microchem, Inc., Newton, Mass.) using a spin-coater (BIDTEC, Freehold, N.J.) at 4000 rpm for 30 s. A 6 μm layer of Shipley 1818 positive photoresist (Microchem) was then spun on in order to mask the PMMA. The resist was exposed through a dark-field chrome mask to UV light using a MJB3 Mask Aligner (Karl Suss, Waterbury Center, Vt.). The patterned areas were developed in a working dilution of Microposit 351 (Microchem) and rinsed in DI water. The unmasked area of PMMA was exposed to oxygen plasma in a reactive ion etcher (PlasmaTherm 790 Series RIE) to transfer the defined pattern into the desired polymer. Any remaining resist was subsequently removed using 1112A remover (Microchem).

Retinal Progenitor Cell Isolation

All experiments were performed according to the Schepens Eye Research Institute Animal Care and Use Committee and the ARVO Statement for the Use of Animals in Ophthalmic and Vision Research. Isolation of RPCs was performed as previously described (Klassen et al., Invest Opthalmol Vis Sci. 2004, 45, 4167-4173). Briefly, retinas were isolated from postnatal day 1 enhanced green fluorescent protein positive (GFP+) transgenic mice (C57BL/6 background). Pooled retina were dissociated by mincing and digested with 0.1% type 1 collagenase (Sigma-Aldrich; St. Louis, Mo.) for 20 minutes. The liberated RPCs were passed through a 100 μm mesh filter, centrifuged at 850 rpm for 3 minutes, re-suspended in culture medium (Neurobasal (NB); Invitrogen-Gibco, Rockville, Md.). containing 2 mM L-glutamine, 100 μg/ml penicillin-streptomycin, 20 ng/ml epidermal growth factor (EGF; Promega, Madison, Wis.) and neural supplement (B27; Invitrogen-Gibco) and plated into culture wells (Multiwell, Becton Dickinson Labware, Franklin Lakes, N.J.) Cells were fed with 2 ml of fresh culture medium on alternating days for 2-3 weeks until RPCs were visible as expanding non-adherent spheres. RPC cultures were passaged 1:5 every 7 days.

Attachment of RPCs to PMMA Scaffolds

PMMA film scaffolds (10×10 mm) were incubated in 70% ethanol for 24 hours and rinsed 3 times with Hanks Buffered Saline Solution (HBSS; Sigma-Aldrich). PMMA scaffolds were then adhered to culture well floors with sterile medical sealant and incubated in 10 μg poly-L-lysine and 100 μg laminin for 10 minutes. Polymers were then rinsed 3 times with HBSS. Cultured GFP+ RPCs were dissociated into single cell suspensions and 1 ml (4×105 cells) were seeded onto each PMMA membrane. The total volume of NB in each well was brought to 2 ml with NB media, in which RPCs were allowed to proliferate on the polymers for 7 days.

Transplantation Surgery

Porous and non-porous PMMA film scaffolds with adherent RPCs were cut into 1×1 mm squares using a McIlwain tissue chopper (Mickle Laboratory Engineering, Gomshell, U.K.) in preparation for transplantation. Mice were placed under general anesthesia with an intraperitoneal injection of ketamine (5 mg/kg) and xylazine (10 mg/kg) and the pupil dilated with 1% tropicamide, topically applied. Mice were placed on a warm heating blanket for surgery. Silk thread (8-0) was used to suture the eyelid open and the eye was stabilized using two 11-0 conjuctival sutures. An incision (˜5 mm) was made in the lateral posterior sclera using a Sharpoint 5.0 mm blade scalpel (Fine Science Tools, Reading, Pa.) While viewing the ocular fundus through a contact lens and an epifluorescent microscope, a 1×1 mm PMMA+RPC graft was inserted through the sclerotomy into the sub-retinal space using #5 Dumont forceps (Fine Science Tools). A single eye from each C57BL/6 wild-type mouse (n=10) was sub-retinally transplanted with a PMMA+RPC composite graft. The scleral incision was closed with an 11-0 nylon suture and all other sutures were removed.

Tissue Preparation and Histology

C57BL/6 mice that received composite grafts were sacrificed after 4 weeks. Engrafted eyes were enucleated, immersion fixed in 4% paraformaldehyde, rinsed 3 times in HBSS and cryoprotected in 10% then 30% sucrose for 12 hours each at 4° C. Eyes were then placed in a cryomold containing optimum cutting temperature (O.C.T) compound. (ProSciTech, Queensland, Australia) and then frozen on dry ice and cryosectioned at 12 μm. Tissue sections were stained with hemotoxalin and eosin and then characterized immunohistochemically. Retinal sections used to analyze GFP+ RPC characterization were blocked in 1% BSA (Sigma-Aldrich) plus 0.2% Triton then incubated with primary antibodies: neurofilament-200 (nf-200, Sigma Aldrich, 1:1000) and nestin (1:1; Developmental Studies Hybridoma Bank, University of Iowa, Iowa City, Iowa), glial fibrillary acidic protein (GFAP) (1:50, Dako Cytomation, Glostrup, Denmark) and recoverin (1:1000, Chemicon, Temecula, Calif.) for 2 hours at 37° C. Samples were then rinsed 3 times with phosphate-buffered saline (PBS) and reacted with species-specific immunoglobulin conjugated to either Cy2 or Cy3 (Zymed Laboratories, Inc., San Francisco, Calif.) for 1 hour at 37° C. Sections were rinsed 3 times with PBS and cover-slipped in PVA-Dabco with DAPI. GFP+ RPCs were imaged to evaluate cell survival and migration into the host retina. Fluorescent imaging was accomplished using a Nikon Eclipse TE800 Microscope equipped with a Spot ISA-CE camera (Diagnostic Instruments, Sterling Heights, Mich.)

### Example 1

### PMMA Scaffold Fabrication

Micropatterned PMMA thin-film scaffolds were fabricated to contain through pores using a dual process of photolithography and reactive ion etching (FIG. 1, panel A). After the etching process, the diameter of the pores was found to be approximately 11 μm in diameter with an interpore distance of 63 μm (FIG. 1, panel B). The thickness of the ultra-thin film scaffold was measured to be approximately 6 μm in thickness by profilometry (Dektak 8, Veeco, Tucson, Ariz.). Unpatterned (non-porous) and micropatterned (porous) ultra-thin film PMMA scaffolds were separated from the silicon wafer in a single sheet by washing in sterile DI water to dissolve the water-soluble lift-off layer.

### Example 2

### RPC Adherence In Vitro

Non-porous PMMA scaffolds maintained their shape and structural integrity in culture with RPCs for the seven day culture period. Following incubation in poly-L-lysine and laminin, non-porous scaffolds retained RPCs in the primary culture well and through a transfer to a second culture well. Porous PMMA scaffolds retained RPCs with a consistency comparable to the non-porous type with no significant differences observed in culture. At one week in vitro, RPCs on both scaffold types proliferated uniformly across the surface forming an even monolayer radiating from relatively evenly spaced neurospheres (FIG. 2, panels A and B). RPCs were found to remain viable when cultured on both non-porous and porous PMMA. There was no observable difference in the survival or proliferation of RPCs when cultured in the presence or absence of 10×10 mm PMMA scaffolds.

### Example 3

### RPC Survival and Adherence after Subretinal Transplantation

Although RPC adherence and survival were nearly identical in culture on both types of scaffolds, transplantation with non-porous scaffolds showed limited RPC retention. During the transplantation process non-porous scaffolds lost the majority of their RPCs. The failure of RPC adhesion and/or survival was established by a nearly complete absence of GFP+ RPC fluorescence upon microscopic examination at four weeks post-implantation. Cryosectioning followed by hemotoxalin and eosin staining allowed for localization of implanted non-porous PMMA scaffolds adjacent to host retinal tissue. Under fluorescent illumination non-porous scaffolds showed no visible GFP+ RPC signal.

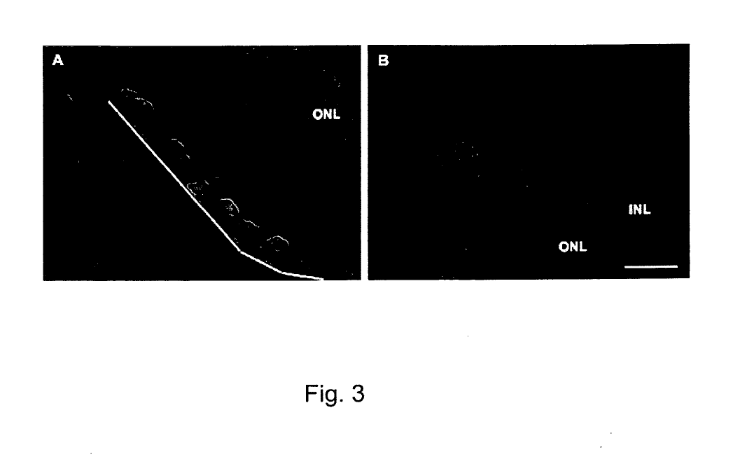

Porous PMMA scaffolds demonstrated consistently higher retention of GFP+ RPCs. The porous topography allowed for RPC adherence through transplantation to the posterior eye for up to four weeks. Under microscopic examination many RPCs appeared closely bound to the porous scaffold and exhibited signs of survival across the entire surface. Enhanced RPC attachment to porous scaffolds further provided a cytoarchtectural microenvironment permissive for eventual cell migration into host retinal layers (FIG. 3, panels A and B).

### Example 4

### RPC Integration and Differentiation after Subretinal Transplantation

Non-porous grafts were associated with RPC integration into the host retina in one out of five transplant recipients. A limited number of RPCs were visibly integrated into host retinal layers in the GFP+ RPC retaining non-porous transplantation (FIG. 4, panel A). The limited integrated RPCs from the non-porous membrane extended relatively short processes and failed to exhibit immunohistochemical markers of retinal differentiation (FIG. 4D). In the remaining four non-porous transplantations there were no GFP+ RPCs visible in any region closely adjacent to or incorporated into host retinal layers.

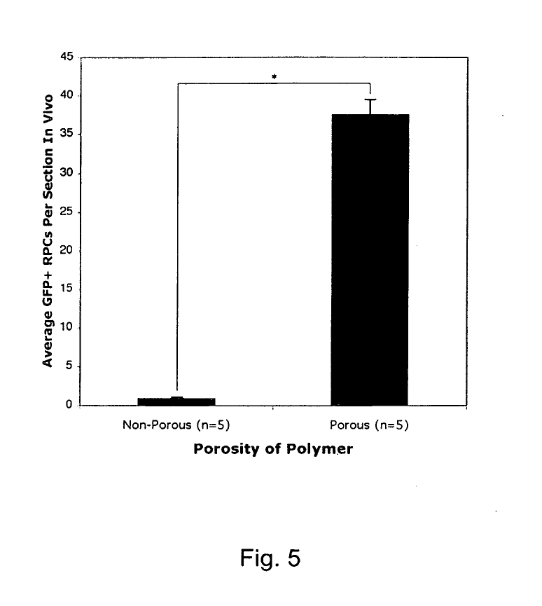

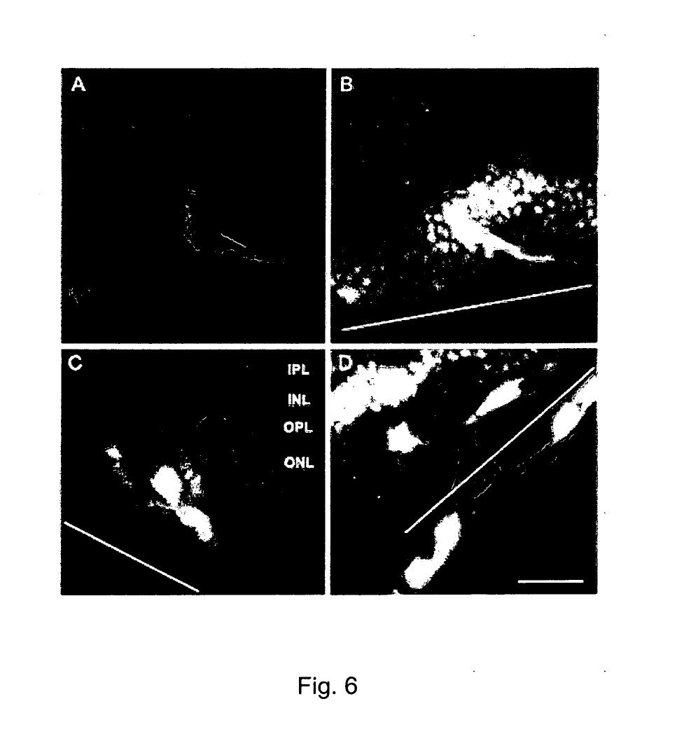

Porous grafts allowed for RPC integration in four out of five transplantations, a four-fold increase when compared to non-porous grafts (FIG. 5). The most robust porous graft transplants exhibited a greater number of integrated GFP+ RPCs (n=80) across host retinal layers (FIG. 4, panels B and C). Initial fluorescent analysis of GFP+ RPCs originating from porous grafts and integration into host retinal layers revealed morphologic differentiation consistent with retinal neurons. In addition, differentiated RPCs in the inner retina developed radial processes similar to those of astrocytes or amacrine cells. Some RPC-derived cells exhibited morphology that spanned the radial extent of the retina, similar to Mueller cells, and these profiles labelled positively for the glial cell marker GFAP (FIG. 4, panels E and F). In the outer retina, integrated RPCs localized to the region of the outer limiting membrane and expressed the retinal-specific protein recoverin (FIG. 6, panels A and B). Recoverin is normally expressed only by photoreceptors and a subset of bipolar cells. RPCs in the earlier stages of migration from porous scaffolds into the outer retina extended processes from scaffolds to retina and expressed the progenitor marker nestin and the early neuronal marker nf-200 (FIG. 6, panels C and D).

The results show that RPCs have similar survival and proliferation rates on micro-machined PMMA and polystryene culture well surfaces in vitro. This is important considering the precarious enterprise of maintaining RPCs in a proliferative state for extended periods of time before transplantation. During the transplantation process a focal retinal detachment is produced at the transplantation site. The ultra-thin (approximately 6 μm) PMMA is well suited for incorporation into this restricted microenvironment. Resolution of retinal detachment is clinically apparent four days post-implantation.

Adhesion of RPCs to porous PMMA during the process of subretinal transplantation demonstrated the utility of a surface topography comprised of 11 μm diameter pores. The average RPC diameter is slightly less than 10 μm. across the surface of a 1 mm graft containing approximately 200 pores provides a mechanism of cell anchorage that involves the insertion of individual cells or their processes into pores. Without being bund to theory, RPCs embedded into pores remain attached to the scaffold during transplantation while also serving as an anchorage point for surrounding RPCs through cell to cell contacts.

Two major advantages to using PMMA with adhesive properties instead of bolus injections for transplantation of RPCs into the subretinal space are increased cell survival and delivery localization to specific retinal regions. Earlier studies attempting to deliver brain-derived neurons into the subretinal space resulted in approximately 90% cell death during the injection process alone (Brundin et al., Nat Med. 2001, 7, 512-513). The use of polymer scaffolds for the delivery of RPCs provides a nine-fold increase in cell survival and a sixteen-fold increase in cell delivery. For the treatment of a retinal degenerative disorder like age-related macular degeneration, where loss of retinal neurons occurs primarily in the macula region, placement of RPC seeded PMMA grafts allows for localized cell replacement.

The many RPCs that remain attached to PMMA scaffolds and reach the sub-retinal space subsequently begin a migratory course towards specific retinal layers. The results show no evidence of RPC migration from the scaffold before 24 hrs. Brain derived precursor cells introduced into the subretinal space have been shown to migrate into host outer retinal layers as early as day 4 post-transplantation (Wojciechowski et al., Glia. 2004, 47, 58-67). By four weeks in vivo approximately 50% of transplanted brain-derived neurons migrate into the host retina (Lu et al., Brain Res. 2002, 943, 292-300). The results provided herein shown migration of similar percentages of RPCs into a range of host retinal layers at 4 weeks post-transplantation. RPCs transplanted on porous PMMA showed the highest percentage of migration into the inner plexiform and outer plexiform layers.

As RPCs reach their target retinal layers, micro-environmental cues stimulate intracellular signaling pathways leading to differentiation. Post-natal RPCs are multipotent and have the potential to differentiate into retinal neurons or glia (Tomita et al., Stem Cells. 2006, 24, 2270-8; Akagi et al., Neuroscie Lett. 2003, 341, 213-6). The present results show markers of neural or glial differentiation at week four in approximately 90% of migrating RPCs. One of the most widely expressed markers indicative of phenotypic maturation was the intermediate filament GFAP. In GFP+ RPCs, the observation of expression of GFAP is indicative of differentiation of the RPCs towards either a retinal astrocytre or Mueller glial cell fate (Zahir et al., Stem Cells. 2005, 23, 424-32). These cell types have many functions in the eye and work directly to facilitate retinal protection and homeostasis.

### Example 5

### Medical Implants Including Biodegradable Nanostructured Surfaces

As a template, an aluminum oxide membrane was used. In general, anodic aluminum oxide films are formed by the electrochemical oxidation of aluminum, as documented in the literature. Depending on the type of anodization process and growth regime used, aluminum oxide membranes can be fabricated to contain nanopores in a wide range of diameters, lengths and interpore distances (Lee et al., Nat. Mater. 2006, 5, 741; Li et al., J. Appl. Phys. 1998, 84, 6023; Masuda et al., Science. 1995, 268, 1466). In order to facilitate nanotube fabrication, here, commercially available aluminum oxide membrane filters were used instead of custom membranes. The anodized aluminum oxide membranes contained pores 20 nm in diameter, 60 μm in length, and a porosity of 10¹¹ pores/cm².

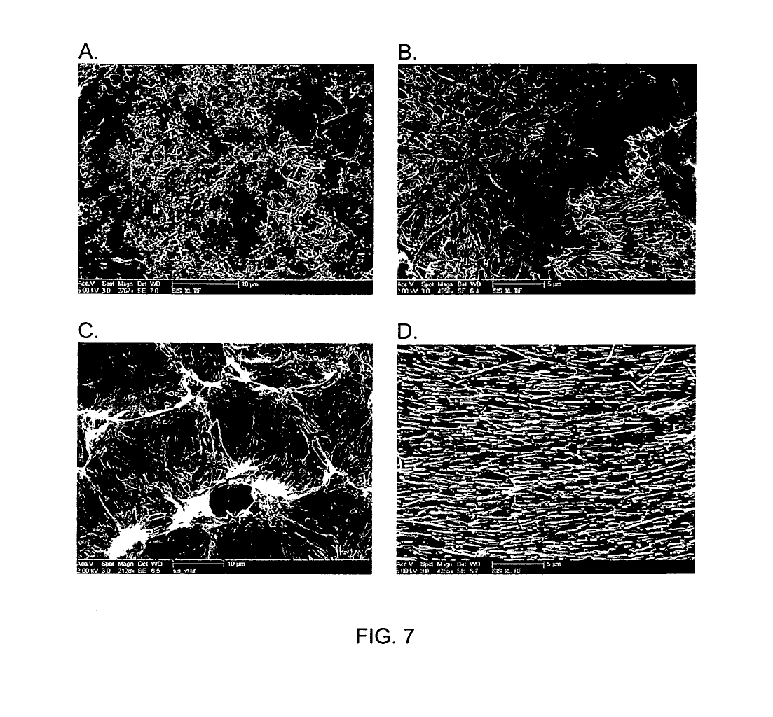

Several biocompatible, biodegradable polymers and composites were utilized for nanostructure formation including 50/50 poly(DL-lactide-co-glycolide) (PLGA) (Amorphous, T_{g}=45 to 50° C.), 25/75 poly(DL-lactide-co-ε-caprolactone) (25/75 DLPLCL) (Amorphous, T_{g}=20° C.), 80/20 poly(DL-lactide-co-ε-caprolactone) (80/20 DLPLCL) (Amorphous, T_{g}=20° C.), and poly(ε-caprolactone) (PCL) (Tₘ=58-63° C., T_{g}=−65 to −60° C.). Polymer melts were formed at 130° C. while in contact with the nanoporous template. After etching the template in a dilute solution of sodium hydroxide, examination by scanning electron microscopy (SEM) showed that nanostructures were be achieved with all of the polymer melts (FIG. 7, panels A-D). However PCL, the only crystalline polymer used, produced the most monodisperse nanotubes. Additionally, the PLGA and PLCL polymers have very high melting points which limit their use in the encapsulation of drugs, especially proteins, by any melt method. Therefore, PCL was used for all further characterization.

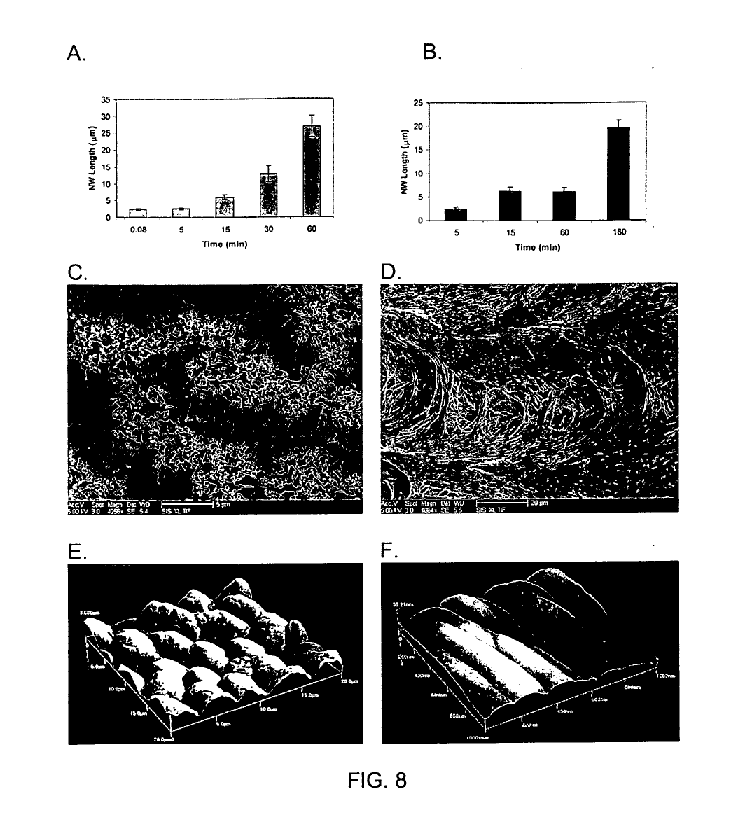

In order to overcome the need for fabricating nanoporous aluminum oxide membranes of varying thickness to produce varying nanotube lengths, nanotube length was instead tuned as a function of melt time and temperature (FIG. 8, panels A and B). At a temperature of 130° C., nanotubes lengths 2.5 to 27.0 μm could be produced in less than 60 minutes by varying contact time. At 65° C. similar structures could be formed, however increase in nanotube length took place over longer intervals. nanotubes less than 10 μm in length, formed at both 65° C. and 130° C., were found to be freestanding (FIG. 8, panel C), though the wires shift to pack in loose clusters as the wires are removed from aqueous solution and dried (FIG. 8, panels C and E). When greater than 10 μm in length, the nanostructures instead folded over to form long strands of arrayed, flexible nanofibers layered over the substrate base (FIG. 8, panels D and F).

No correlation was observed between nanotube diameter and contact time at either temperature. At 130° C. nanotube diameters of 196.1±60.0 nm were observed and at 65° C. diameters of 168.1±39.1 nm. The difference between the nanotube diameters at the two melting points is statistically significant (one-way ANOVA, p=5.2×10⁻⁵) showing that nanotube diameter can be regulated to a certain degree by increasing melt temperature. In both cases, the average nanotube diameter is considerably larger than the template pore size. SEM analysis of the template membrane showed a range of pore diameters with an average diameter of 29.0±9.0 nm, larger than the listed pore diameter though not significant enough to explain the increase in diameter over theoretical values. A similar increase in nanofiber diameter over pore size has been shown elsewhere (Li et al., J. Appl. Polym. Sci. 2006, 99, 1018; Steinhart et al., Angew. Chem. Int. Ed. 2004, 43, 1334). It has been suggested that when the diameter of the template pore is smaller than the thickness of the polymer melt traveling along the inside of the pore (typically 10-30 nm), a massive nanotube will form (Steinhart et al., Angew. Chem. Int. Ed. 2004, 43, 1334). In this particular situation where the pore diameter is 20 nm, it is possible the polymer melt cannot be confined within the volume of the pore. And therefore, as the polymer expands during the melt, a larger sub-micron wire, rather than nanotube, is created during the template process.

Surface roughness of the PCL nanotubes and nanofibers was subsequently examined using atomic force microscopy (AFM). In order to capture a representative sample of the topography, 20 μm scan size intermittent contact images were analyzed. As expected, the formation of nanotubes on the surface of the PCL increased roughness of the substrate (Table 1). Both average roughness (Rₐ) and root mean square roughness (R_{q}) increase with nanotube length. The change in wettability due to the surface roughness was investigated using contact angle measurement (Table 1).

TABLE 1
Surface Roughness Parameters

| NW Length (μm) | Rₐ (nm) | R_{q} (nm) | θ (°) | τ (dynes/cm) |
|---|---|---|---|---|
| 0ₐ | — | — | 60.3 ± 4.5 | 51.7 ± 2.5 |
| 0_{b} | 5.9 | 7.7 | 66.5 ± 3.8 | 48.2 ± 2.7 |
| 2.5 | 710.3 | 822.2 | 70.0 ± 2.0 | 45.7 ± 1.5 |
| 27 | 1357 | 1819 | 70.7 ± 4.2 | 45.0 ± 2.6 |

Legend:
[a] 0ₐ = PCL,
0_{b} = Heat,
NaOH treated PCL,
Rₐ = Average roughness (20 μm scan size),
R_{q} = Root mean square roughness (20 μm scan size),
θ = Water contact angle,
τ = Wetting tension.

As shown in Table 1, contact angles for solvent cast PCL and PCL treated with heat and exposure to sodium hydroxide etchant were not significantly different. Disks of PCL nanotubes, however, were found to have an initial contact angle of approximately 16° (nanotubes measured less than 20 μm in length) or 32° (nanotubes measured greater than 20 μm in length). After dewetting, the disks reached an equilibrium contact angle measurement of approximately 70.3±2.9°, regardless of nanotube length. The heat treatment and exposure to sodium hydroxide did not significantly change the wettability of the PCL, however, the surface roughness of both the free-standing nanotubes and flexible nanofibers formed by template synthesis decreased the wettability of the surface (one-way ANOVA, p=0.007, post-hoc Tukey's test).

Different models have been proposed to understand the affect of surface roughness on contact angle. The “wetted surface” model assumes that the liquid fills the depressions in the region where it contacts the rough surface, (Wenzel, T.N. J. Phys. Colloid Chem. 1949, 1466) whereas the “composite surface” model dictates that the liquid is lifted up by the roughness features (Cassie, A. B. D. Discuss. Faraday Soc. 1948, 3, 11). According to the wetted surface model, the contact angle on arrays of 2.5 μm and 27 μm nanotubes fabricated from the aluminum oxide membrane template will both approach approximately 90.0°. According to the composite surface model, both nanotube arrays should approach a contact angle of 94.4°. Several groups have used experimental results to correlate surface roughness of patterned materials, such as nanotubes, with wetted or composite contact angles (Parthasarathy et al., Chem. Mater. 1994, 6, 1627; Bico et al., Europhys Lett. 2001, 55, 214; He et al., Langmuir. 2003, 19, 4999; Patankar et al., Langmuir. 2003, 19, 1249) and it has been suggested that generally surfaces exhibiting hydrophilic properties (θ<90° will follow the wetted surface model for roughness whereas surfaces exhibiting hydrophobic properties (θ>90° will follow the composite surface model though either can be induced by physical manipulation.

The difference between the experimental and theoretical values here for contact angle can be attributed to the tight packing of the nanotubes as well as the nanotube diameter (Parthasarathy et al., Chem. Mater. 1994, 6, 1627). Here, the compact packing of the nanotubes (interpore distance <20 nm) does not allow either water or air to be trapped between the projections. In the case where distance between wires is negligibly close, the ratio of the rough surface area to projected area (r) approaches closer to 1. The increase in diameter of the PCL nanotube in comparison to the template pore size causes the contact area with the liquid to increase, and therefore the ratio of contact area to the rough surface area (fₛ) also approaches closer to 1. Similarly, in the case of the nanofiber arrays, the folded over nanofibers cause a decrease in the projected surface area (as the wires are no longer free standing), as well as an increase in the liquid contact area. Therefore the wettability of the nanostructured PCL remains relatively similar to smooth PCL rather than forming super hydrophobic or super hydrophilic surfaces.

As a biocompatible and biodegradable polymer, PCL has been investigated for the controlled delivery of low molecular weight drugs. PCL is known to be highly permeable, though insoluble in water (Bodmeier et al., Control. Release. 1989, 10, 167; Livshits et al., Pharm. Chem. J. 1988, 22, 515; Pitt et al., J. Biomed. Mater. Res. 1979, 13, 497; Tarvainen et al., Eur. J. Pharm. Sci. 2002, 16, 323). Combined with its high crystallinity and low degradation rate, PCL is well-suited for implantable, long term drug delivery systems. Potential therapeutic substances such as proteins and peptides may be encapsulated in PCL in the absence of denaturing organic solvents by way of hot-melt encapsulation. Previous studies have shown that melt encapsulation of proteins within PCL produces feasible systems for controlled delivery of stable proteins (Jameela et al., J. Biomater.s Sci. Polym. Ed. 1997, 8, 457; Lin et al., J. Microencapsul. 2001, 18, 585).

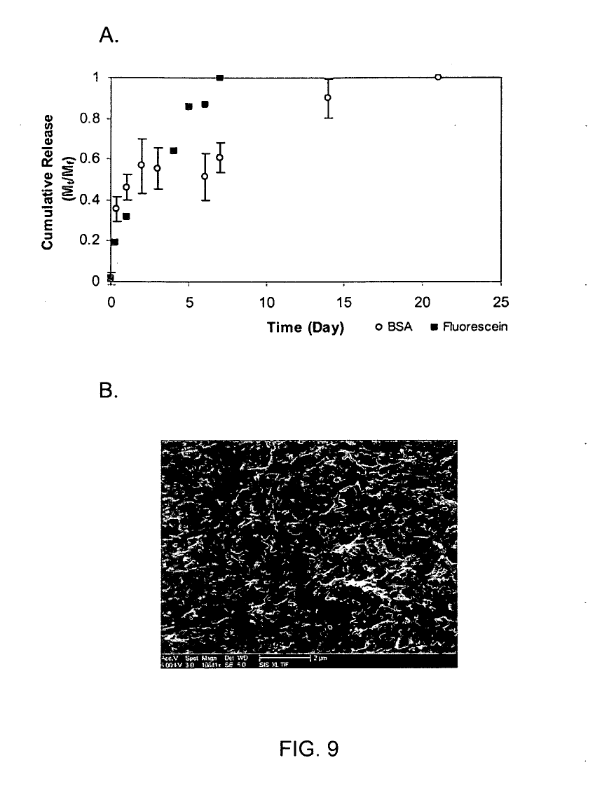

The method of template synthesis with a PCL polymer melt not only provides a means to fabricate nanotube arrays, but also a potential means to incorporate protein molecules in the nanotubes without the use of organic solvents. Here, fluorescein (MW=389.38 Da, R_{H}=6.5 Å, R_{G}=9.8 Å) was used as a small molecule model and bovine serum albumin (BSA) (MW=66 kDa, R_{H}=3.7 nm, R_{G}=2.87 nm) as a model protein for encapsulation and release from PCL nanotubes (6 μm in length). Fluorescein was found to release steadily over a period of a week (FIG. 9, panel A). A linear regression analysis of the log percent drug released versus log time was performed using the first 60% of the release curve to obtain the diffusional exponent (Peppas, N. A. Pharm. Acta Hely. 1985, 60, 110). The diffusional exponent (n) for release of fluorescein from the PCL nanotube substrate (n=0.46, R²=0.997) was found to fall in the range 0.43<n<0.5, indicating release controlled by Fickian diffusion (Peppas, N. A. Pharm. Acta Helv. 1985, 60, 110). The release profile for BSA, however, was characterized as a short burst phase during the first 8 hours where approximately 30% of the cumulative protein released was delivered. Sustained release was achieved over a period of 21 days after which release leveled off and no additional protein was released (FIG. 9, panel A). The diffusional exponent for release of BSA from the PCL nanotube substrate (n=0.26, R²=0.997) was found to be less than 0.5.

It has been previously suggested that drug release from a system with a diffusional exponent less than 0.5 may be due to a combination of diffusion through the matrix and partial diffusion through water filled pores (Tarvainen et al., Eur. J. Pharm. Sci. 2002, 16, 323; Peppas, N. A. Pharm. Acta Helv. 1985, 60, 110). Therefore, the initial burst in protein release was most likely due to drug desorption at the nanotube surface where the surrounding fluid is able to begin dissolving exposed protein immediately. Whereas the diffusional release phase is attributed to molecule diffusion from near the surface. As the protein at the surface is dissolved, the porosity of the PCL nanotubes is increased, allowing fluid to dissolve protein molecules embedded in the nanotube. Protein molecules fully coated and embedded within the nanotube, however, are gradually released due to hindrance of diffusion of BSA from the inner part of the nanotube. The morphology of the PCL nanotubes after degradation in PBS over 7 weeks was observed using SEM. After degradation, the nanotube morphology is less apparent; the nanotube are matted together or degraded completely from the surface (FIG. 9, panel B).

The results provided herein show that retinal progenitor cells can be combined with polymer substrates for the generation of tissue equivalents in culture. RPCs delivered using these polymers send projections into the host retina and express at least three markers appropriate to this tissue. A porous PMMA topography is beneficial for adherence of RPCs delivered in vivo. Therefore, the results show that ultra-thin PMMA scaffolds provide a suitable cytoarchitectural environment for tissue engineering and transplantation to the diseased eye.

What is claimed is:

1. A medical implant, comprising:
a cell contacting surface or film comprising nanotopography of nanofibers, nanotubes, nanochannels, microchannels or microwells, wherein said nanochannels and microchannels comprise a first and second opening at lateral edges of said cell contacting surface or film, and
wherein said nanotopography is capable of enhancing or promoting cell differentiation or cell viability at said cell contacting surface or film.

2. The medical implant of claim 1, wherein said medical implant is an orthopedic implant, a dental implant, a cardiovascular implant, a neurological implant, a neurovascular implant, a gastrointestinal implant, a muscular implant, or an ocular implant.

3. The medical implant of claim 1, wherein said cell contacting surface or film expands or unfurls in the presence of a hydrating liquid.

4. The medical implant of claim 1, wherein said nanotopography is comprised of poly(ε-caprolactone) (PCL).

5. The medical implant of claim 1, wherein said nanotopography is comprised of poly(DL-lactide-co-glycolide) (PLGA), poly(DL-lactide-co-ε-caprolactone) (DLPLCL), poly(ε-caprolactone) (PCL), collogen, gelatin, agarose, poly(methyl methacrylate), galatin/ε-caprolactone, collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above, or combinations thereof.

6. The medical implant of claim 1, wherein said nanotopography is comprised of poly(methyl methacrylate).

7. The medical implant of claim 1, wherein said nanotopography is comprised of silicon, titania, zirconia, cobalt-chromium, alumina, silica, barium aluminate, barium titanate, iron oxide, and zinc oxide, or combinations thereof.

8. The medical implant of claim 1, wherein said nanotopography further comprises an agent to facilitate cell adhesion and cell growth selected from the group consisting of laminin, fibrin, fibronectin, proteoglycans, glycoproteins, glycosaminoglycans, chemotactic agents, and growth factors.

9. The medical implant of claim 1, wherein the nanotopography further comprise a bioactive agent for elution to surrounding tissue upon placement of said implant in subject.

10. The medical implant of claim 9, wherein said bioactive agent is selected from a growth factor, a steroid agent, an antibody therapy, an antimicrobial agent, an antibiotic, an antiretroviral drug, an anti-inflammatory compound, an antitumor agent and a chemotherapeutic agent.

11. The medical implant of claim 1, wherein said nanotopography is capable of limiting cell adhesion and cell growth.

12. The medical implant of claim 1, wherein said nanofibers or nanotubes range in length from about 1 μm to about 70 μm.

13. The medical implant of claim 1, wherein said nanofibers or nanotubes range in diameter from about 3 nm to about 300 nm.

14. The medical implant of claim 13, wherein said nanotopography comprises nanofibers at a density greater than 100,000,000 nanofibers per square centimeter.

15. The medical implant of claim 13, wherein said nanotopography comprises nanotubes at a density greater than 25,000,000 nanotubes per square centimeter.

16. The medical implant of claim 1, wherein said nanotubes have a pore diameter range from about 3 nm to about 250 nm.

17. The medical implant of claim 1, wherein said nanotopography ranges in thickness from about 1 μm to about 100 μm.

18. The medical implant of claim 1, wherein said nanotopography ranges in thickness from about 2 μm to about 20 μm.

19. The medical implant of claim 1, wherein said microwells range in diameter from about 5 μm to about 12 μm.

20. The medical implant of claim 19, wherein said nanotopography comprises microwells at a density greater than 150,000 microwells per square centimeter.

21. The medical implant of claim 1, wherein said nanochannels range in diameter from about mm to about 1000 nm.

22. The medical implant of claim 21, wherein said nanotopography comprises nanochannels at a density greater than 25,000,000 nanochannels per square centimeter.

23. The medical implant of claim 1, wherein said microchannels range in diameter from about 1 μm to about 500 μm.

24. The medical implant of claim 23, wherein said nanotopography comprises microchannels at a density greater than 150,000 microchannels per square centimeter.

25. The medical implant of claim 1, wherein said nanotopography further comprises cells.

26. The medical implant of claim 25, wherein said cell is a stem cell, a retinal progenitor cell, or a neuronal cell.

27.-123. (canceled)
